(19) United States
(12) Patent Application Publication
(10) Pub. No.: US 2015/0320914 A1
(43) Pub. Date: Nov. 12, 2015

(54) STENTS HAVING BIODEGRADABLE LAYERS

(71) Applicant: Micell Technologies, Inc., Durham, NC (US)

(72) Inventors: James B. McClain, Raleigh, NC (US); Charles Douglas Taylor, Franklinton, NC (US)

(21) Appl. No.: 14/716,975

(22) Filed: May 20, 2015

Related U.S. Application Data
(63) Continuation of application No. 12/595,848, filed on Mar. 26, 2010, now abandoned.
(60) Provisional application No. 60/981,445, filed on Oct. 19, 2007.
(60) Provisional application No. 60/912,394, filed on Apr. 17, 2007.
(60) Provisional application No. 60/912,408, filed on Apr. 17, 2007.

(51) Int. Cl. A61L 31/10; A61L 31/14; A61F 2/82; A61L 31/16

(52) U.S. Cl. CPC A61L 31/10; A61L 31/16; A61L 31/148; A61F 2/82; A61L 2420/02; A61L 2420/08; A61F 2230/0069

(57) ABSTRACT

Provided herein is a coated coronary stent, comprising: a. stent framework; b. a plurality of layers deposited on said stent framework to form said coronary stent; wherein at least one of said layers comprises a bioabsorbable polymer and at least one of said layers comprises one or more active agents; wherein at least part of the active agent is in crystalline form.

### CROSS REFERENCE TO RELATED APPLICATIONS

This application is a continuation of U.S. application Ser. No. 12/595,848 filed on Mar. 26, 2010, which application was filed pursuant to 35 U.S.C. §371 as a United States National Phase Application of International Application No. PCT/US2008/060671 entitled “Stents Having Biodegradable Layers”, filed on Apr. 17, 2008, which claims the benefit of U.S. Provisional Application No. 60/912,408, filed Apr. 17, 2007, U.S. Provisional Application No. 60/912,394, filed Apr. 17, 2007, and U.S. Provisional Application No. 60/981,445, filed Oct. 19, 2007. The contents of each of these applications are incorporated herein by reference in their entirety.

### BACKGROUND OF THE INVENTION

The present invention relates to methods for forming stents comprising a bioabsorbable polymer and a pharmaceutical or biological agent in powder form onto a substrate.

It is desirable to have a drug-eluting stent with minimal physical, chemical and therapeutic legacy in the vessel after a proscribed period of time. This period of time is based on the effective healing of the vessel after opening the blockage by PCI/stenting (currently believed by leading clinicians to be 6-18 months).

It is also desirable to have drug-eluting stents of minimal cross-sectional thickness for (a) flexibility of deployment (b) access to small vessels (c) minimized intrusion into the vessel wall and blood.

### SUMMARY OF THE INVENTION

One embodiment provides a coated coronary stent, comprising: a stent framework and a rapamycin-polymer coating wherein at least part of rapamycin is in crystalline form and the rapamycin-polymer coating comprises one or more resorbable polymers.

In another embodiment the rapamycin-polymer coating has substantially uniform thickness and rapamycin in the coating is substantially uniformly dispersed within the rapamycin-polymer coating.

In another embodiment, the one or more resorbable polymers are selected from PLGA (poly(lactide-co-glycolide); DLPLA—poly(dl-lactide); LPLA—poly(1-lactide); PGA—polyglycolide; PDO—poly(dioxanone); PGA-TMC—poly(glycolide-co-trimethylene carbonate); PGA-LPLA—poly(1-lactide-co-glycolide); PGA-DLPLA—poly(dl-lactide-co-glycolide); LPLA-DLPLA—poly(1-lactide-co-dl-lactide); PDO-PGA-TMC—poly(glycolide-co-trimethylene carbonate-co-dioxanone) and combinations thereof.

In yet another embodiment the polymer is 50/50 PLGA.

In still another embodiment the at least part of said rapamycin forms a phase separate from one or more phases formed by said polymer.

In another embodiment the rapamycin is at least 50% crystalline.

In another embodiment the rapamycin is at least 75% crystalline.

In another embodiment the rapamycin is at least 90% crystalline.

In another embodiment the rapamycin is at least 95% crystalline.

In another embodiment the rapamycin is at least 99% crystalline.

In another embodiment the polymer is a mixture of two or more polymers.

In another embodiment the mixture of polymers forms a continuous film around particles of rapamycin.

In another embodiment the two or more polymers are intimately mixed.

In another embodiment the mixture comprises no single polymer domain larger than about 20 nm.

In another embodiment the each polymer in said mixture comprises a discrete phase.

In another embodiment the discrete phases formed by said polymers in said mixture are larger than about 10 nm.

In another embodiment the discrete phases formed by said polymers in said mixture are larger than about 50 nm.

In another embodiment the rapamycin in said stent has a shelf stability of at least 3 months.

In another embodiment the rapamycin in said stent has a shelf stability of at least 6 months.

In another embodiment the rapamycin in said stent has a shelf stability of at least 12 months.

In another embodiment the coating is substantially conformal.

In another embodiment the stent provides an elution profile wherein about 10% to about 50% of rapamycin is eluted at week 1 after the composite is implanted in a subject under physiological conditions, about 25% to about 75% of rapamycin is eluted at week 2 and about 50% to about 100% of rapamycin is eluted at week 6.

In another embodiment the stent provides an elution profile wherein about 10% to about 50% of rapamycin is eluted at week 1 after the composite is implanted in a subject under physiological conditions, about 25% to about 75% of rapamycin is eluted at week 2 and about 50% to about 100% of rapamycin is eluted at week 10.

In another embodiment the stent framework is a stainless steel framework.

Still another embodiment provides a coated coronary stent, comprising: a stent and a macrolide immunosuppressive (limus) drug-polymer coating wherein at least part of the drug is in crystalline form and the macrolide immunosuppressive—polymer coating comprises one or more resorbable polymers.

In another embodiment the macrolide immunosuppressive drug comprises one or more of rapamycin, 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin, 40-O-(2-Hydroxyl)ethoxycarbonylmethyl-rapamycin, 40-O-(3-Hydroxyl)propyl-rapamycin, 40-O-(6-Hydroxyl)hexyl-rapamycin, 40-O-[2-(2-Hydroxyl)ethoxy]ethyl-rapamycin, 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin, 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxyl)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), and 42-[3-hydroxy-2-(hydroxymethyl)-2-methylpropanoate]rapamycin (temsirolimus).

In another embodiment the macrolide immunosuppressive drug is at least 50% crystalline.

Another embodiment provides a method for preparing a coated coronary stent comprising forming a macrolide immunosuppressive (limus) drug-polymer coating on the stent framework wherein at least part of the drug is in crystalline form and the macrolide immunosuppressive—polymer coating comprises one or more resorbable polymers.

The present invention provides several advantages which overcome or attenuate the limitations of current technology for bioabsorbable stents.

One embodiment provides a coated coronary stent, comprising: a stent framework and a rapamycin-polymer coating wherein at least part of rapamycin is in crystalline form and the rapamycin-polymer coating comprises one or more resorbable polymers.

In another embodiment the rapamycin-polymer coating has substantially uniform thickness and rapamycin in the coating is substantially uniformly dispersed within the rapamycin-polymer coating.

In another embodiment, the one or more resorbable polymers are selected from PLGA (poly(lactide-co-glycolide); DLPLA—poly(dl-lactide); LPLA—poly(1-lactide); PGA—polyglycolide; PDO—poly(dioxanone); PGA-TMC—poly(glycolide-co-trimethylene carbonate); PGA-LPLA—poly(1-lactide-co-glycolide); PGA-DLPLA—poly(dl-lactide-co-glycolide); LPLA-DLPLA—poly(1-lactide-co-dl-lactide); PDO-PGA-TMC—poly(glycolide-co-trimethylene carbonate-co-dioxanone) and combinations thereof.

Another embodiment provides a method for preparing a coated coronary stent comprising the following steps: providing a stainless or cobalt—chromium stent framework; forming a macrolide immunosuppressive (limus) drug-polymer coating on the stent framework wherein at least part of the drug is in crystalline form and the polymer is bioabsorbable.

In another embodiment the macrolide is deposited in dry powder form.

In another embodiment the bioabsorbable polymer is deposited in dry powder form.

In another embodiment the polymer is deposited by an e-SEDS process.

In another embodiment the polymer is deposited by an e-RESS process.

Another embodiment provides a method further comprising sintering said coating under conditions that do not substantially modify the morphology of said macrolide.

Yet another embodiment provides a coated coronary stent, comprising: a stent framework a first layer of bioabsorbable polymer; and a rapamycin-polymer coating comprising rapamycin and a second bioabsorbable polymer wherein at least part of rapamycin is in crystalline form and wherein the first polymer is a slow absorbing polymer and the second polymer is a fast absorbing polymer.

Yet another embodiment provides a coated coronary stent, comprising: a stent framework; a first layer of bioabsorbable polymer; and a rapamycin-polymer coating comprising rapamycin and a second bioabsorbable polymer wherein at least part of rapamycin is in crystalline form and wherein the first polymer is a slow absorbing polymer and the second polymer is a fast absorbing polymer.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized and the accompanying drawings of which:

FIG. 1 depicts an image of an embodiment of a coated stent.

FIG. 2 depicts the process steps of an embodiment of coating a substrate.

FIG. 3 depicts a cross-sectional Scanning Electron Microscope image of Rapamycin/PEVA/PBMA coated stents at ×7000 magnification. Four cross-sectional thicknesses of the coating, 101, on the stent framework, 100, were measured: (1) 10.355 μM; (2) 10.412 μM; (3) 10.043 μM and (4) 10.157 μM, providing a calculated average thickness of 10.242 μM±2%.

FIG. 4 depicts the ability of the coatings of the present methods and devices produced thereby to load drug at intended locations in the device.

FIG. 5 shows a Drug-Polymer coated coronary stent (a) immediately after deposition, (b) after annealing in a dense carbon dioxide environment at 40° C.

FIG. 6 shows 40× Magnified Images of Rapamycin/PEVA/PBMA Coated Stents, Obtained From an Optical Microscope with Back and Side Lighting, Showing the Outside, Edge and Inside Surfaces, (a) before and (b) after sintering provides a description of the technology of an embodiment provided herein.

FIG. 7 shows 40× Magnified Images of Rapamycin/PEVA/PBMA Coated Stents, Obtained From an Optical Microscope with Back and Side Lighting, Showing the Outside and Inside Surfaces, (a) before and (b) after sintering

FIG. 8 shows 100× Magnified Image of a Rapamycin/PEVA/PBMA Coated Stent, Obtained from an Optical Microscope. Crystalline drug is clearly visible embedded within a highly uniform polymer coating.

FIG. 9 shows Scanning Electron Microscope Images of Rapamycin/PEVA/PBMA Coated Stents, at (a) ×30 magnification, (b) ×250 magnification, (c) ×1000 magnification and (d) ×3000 magnification.

FIG. 10 depicts Rapamycin Elution Profile of coated stents (PLGA/Rapamycin coatings) where the elution profile was determined by static elution media of 5% EtOH/water, pH 7.4, 37° C. as described in Example 3 of coated stents described therein.

FIG. 11 depicts Rapamycin Elution Rates of coated stents (PLGA/Rapamycin coatings) where the static elution profile was compared with agitated elution profile by an elution media of 5% EtOH/water, pH 7.4, 37° C. as described in Example 3 of coated stents described therein

FIG. 12 depicts Rapamycin Elution Profile of coated stents (PLGA/Rapamycin coatings) where the elution profile by 5% EtOH/water, pH 7.4, 37° C. elution buffer was compared with the elution profile using phosphate buffer saline pH 7.4, 37° C.; both profiles were determined as described in Example 3 of coated stents described therein.

### DETAILED DESCRIPTION OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations and variations thereof.

### DEFINITIONS

As used in the present specification, the following words and phrases are generally intended to have the meanings as set forth below, except to the extent that the context in which they are used indicates otherwise.

“Substrate” as used herein, refers to any surface upon which it is desirable to deposit a coating comprising a polymer and a pharmaceutical or biological agent, wherein the coating process does not substantially modify the morphology of the pharmaceutical agent or the activity of the biological agent. Biomedical implants are of particular interest for the present invention; however the present invention is not intended to be restricted to this class of substrates. Those of skill in the art will appreciate alternate substrates that could benefit from the coating process described herein, such as pharmaceutical tablet cores, as part of an assay apparatus or as components in a diagnostic kit (e.g. a test strip).

“Biomedical implant” as used herein refers to any implant for insertion into the body of a human or animal subject, including but not limited to stents (e.g., vascular stents), electrodes, catheters, leads, implantable pacemaker, cardioverter or defibrillator housings, joints, screws, rods, ophthalmic implants, femoral pins, bone plates, grafts, anastomotic devices, perivascular wraps, sutures, staples, shunts for hydrocephalus, dialysis grafts, colostomy bag attachment devices, ear drainage tubes, leads for pace makers and implantable cardioverters and defibrillators, vertebral disks, bone pins, suture anchors, hemostatic barriers, clamps, screws, plates, clips, vascular implants, tissue adhesives and sealants, tissue scaffolds, various types of dressings (e.g., wound dressings), bone substitutes, intraluminal devices, vascular supports, etc.

The implants may be formed from any suitable material, including but not limited to organic polymers (including stable or inert polymers and biodegradable polymers), metals, inorganic materials such as silicon, and composites thereof, including layered structures with a core of one material and one or more coatings of a different material. Substrates made of a conducting material facilitate electrostatic capture. However, the invention contemplates the use of electrostatic capture in conjunction with substrate having low conductivity or which non-conductive. To enhance electrostatic capture when a non-conductive substrate is employed, the substrate is processed while maintaining a strong electrical field in the vicinity of the substrate.

Subjects into which biomedical implants of the invention may be applied or inserted include both human subjects (including male and female subjects and infant, juvenile, adolescent, adult and geriatric subjects) as well as animal subjects (including but not limited to dog, cat, horse, monkey, etc.) for veterinary purposes.

In a preferred embodiment the biomedical implant is an expandable intraluminal vascular graft or stent (e.g., comprising a wire mesh tube) that can be expanded within a blood vessel by an angioplasty balloon associated with a catheter to dilate and expand the lumen of a blood vessel, such as described in U.S. Pat. No. 4,733,665 to Palmaz Shaz.

“Pharmaceutical agent” as used herein refers to any of a variety of drugs or pharmaceutical compounds that can be used as active agents to prevent or treat a disease (meaning any treatment of a disease in a mammal, including preventing the disease, i.e. causing the clinical symptoms of the disease not to develop; inhibiting the disease, i.e. arresting the development of clinical symptoms; and/or relieving the disease, i.e. causing the regression of clinical symptoms). It is possible that the pharmaceutical agents of the invention may also comprise two or more drugs or pharmaceutical compounds. Pharmaceutical agents, include but are not limited to antirestenotic agents, antidiabetics, analgesics, antiinflammatory agents, antirheumatics, antihypotensive agents, antihypertensive agents, psychoactive drugs, tranquillizers, antiemetics, muscle relaxants, glucocorticoids, agents for treating ulcerative colitis or Crohn's disease, antiallergics, antibiotics, antiepileptics, anticoagulants, antimycotics, antitussives, arteriosclerosis remedies, diuretics, proteins, peptides, enzymes, enzyme inhibitors, gout remedies, hormones and inhibitors thereof, cardiac glycosides, immunotherapeutic agents and cytokines, laxatives, lipid-lowering agents, migraine remedies, mineral products, otologicals, anti parkinson agents, thyroid therapeutic agents, spasmolytics, platelet aggregation inhibitors, vitamins, cytostatics and metastasis inhibitors, phytopharmaceuticals, chemotherapeutic agents and amino acids. Examples of suitable active ingredients are acarbose, antigens, beta-receptor blockers, non-steroidal antiinflammatory drugs {NSAIDs], cardiac glycosides, acetylsalicylic acid, virustatics, aclarubicin, acyclovir, cisplatin, actinomycin, alpha- and beta-sympatomimetics, (dmeprazole, allopurinol, alprostadil, prostaglandins, amantadine, ambroxol, amlodipine, methotrexate, S-aminosalicylic acid, amitriptyline, amoxicillin, anastrozole, atenolol, azathioprine, balsalazide, beclomethasone, betahistine, bezafibrate, bicalutamide, diazepam and diazepam derivatives, budesonide, bufexamac, buprenorphine, methadone, calcium salts, potassium salts, magnesium salts, candesartan, carbamazepine, captopril, cefalosporins, cetirizine, chenodeoxycholic acid, ursodeoxycholic acid, theophylline and theophylline derivatives, trypsins, cimetidine, clarithromycin, clavulanic acid, clindamycin, clobutinol, clonidine, cotrimoxazole, codeine, caffeine, vitamin D and derivatives of vitamin D, colestyramine, cromoglicic acid, coumarin and coumarin derivatives, cysteine, cytarabine, cyclophosphamide, ciclosporin, cyproterone, cytabarine, dapiprazole, desogestrel, desonide, dihydralazine, diltiazem, ergot alkaloids, dimenhydrinate, dimethyl sulphoxide, dimeticone, domperidone and domperidan derivatives, dopamine, doxazosin, doxorubizin, doxylamine, dapiprazole, benzodiazepines, diclofenac, glycoside antibiotics, desipramine, econazole, ACE inhibitors, enalapril, ephedrine, epinephrine, epoetin and epoetin derivatives, morphinans, calcium antagonists, irinotecan, modafinil, orlistat, peptide antibiotics, phenytoin, riluzoles, risedronate, sildenafil, topiramate, macrolide antibiotics, oestrogen and oestrogen derivatives, progestogen and progestogen derivatives, testosterone and testosterone derivatives, androgen and androgen derivatives, ethenzamide, etofenamate, etofibrate, fenofibrate, etofylline, etoposide, famciclovir, famotidine, felodipine, fenofibrate, fentanyl, fenticonazole, gyrase inhibitors, fluconazole, fludarabine, fluarizine, fluorouracil, fluoxetine, flurbiprofen, ibuprofen, flutamide, fluvastatin, follitropin, formoterol, fosfomicin, furosemide, fusidic acid, gallopamil, ganciclovir, gemfibrozil, gentamicin, ginkgo, Saint John's wort, glibenclamide, urea derivatives as oral antidiabetics, glucagon, glucosamine and glucosamine derivatives, glutathione, glycerol and glycerol derivatives, hypothalamus hormones, goserelin, gyrase inhibitors, guanethidine, halofantrine, haloperidol, heparin and heparin derivatives, hyaluronic acid, hydralazine, hydrochlorothiazide and hydrochlorothiazide derivatives, salicylates, hydroxyzine, idarubicin, ifosfamide, imipramine, indometacin, indoramine, insulin, interferons, iodine and iodine derivatives, isoconazole, isoprenaline, glucitol and glucitol derivatives, itraconazole, ketoconazole, ketoprofen, ketotifen, lacidipine, lansoprazole, levodopa, levomethadone, thyroid hormones, lipoic acid and lipoic acid derivatives, lisinopril, lisuride, lofepramine, lomustine, loperamide, loratadine, maprotiline, mebendazole, mebeverine, meclozine, mefenamic acid, mefloquine, meloxicam, mepindolol, meprobamate, meropenem, mesalazine, mesuximide, metamizole, metformin, methotrexate, methylphenidate, methylprednisolone, metixene, metoclopramide, metoprolol, metronidazole, mianserin, miconazole, minocycline, minoxidil, misoprostol, mitomycin, mizolastine, moexipril, morphine and morphine derivatives, evening primrose, nalbuphine, naloxone, tilidine, naproxen, narcotine, natamycin, neostigmine, nicergoline, nicethamide, nifedipine, niflumic acid, nimodipine, nimorazole, nimustine, nisoldipine, adrenaline and adrenaline derivatives, norfloxacin, novamine sulfone, noscapine, nystatin, ofloxacin, olanzapine, olsalazine, omeprazole, omoconazole, ondansetron, oxaceprol, oxacillin, oxiconazole, oxymetazoline, pantoprazole, paracetamol, paroxetine, penciclovir, oral penicillins, pentazocine, pentifylline, pentoxifylline, perphenazine, pethidine, plant extracts, phenazone, pheniramine, barbituric acid derivatives, phenylbutazone, phenytoin, pimozide, pindolol, piperazine, piracetam, pirenzepine, piribedil, piroxicam, pramipexole, pravastatin, prazosin, procaine, promazine, propiverine, propranolol, propyphenazone, prostaglandins, protionamide, proxyphylline, quetiapine, quinapril, quinaprilat, ramipril, ranitidine, reproterol, reserpine, ribavirin, rifampicin, risperidone, ritonavir, ropinirole, roxatidine, roxithromycin, ruscogenin, rutoside and rutoside derivatives, sabadilla, salbutamol, salmeterol, scopolamine, selegiline, sertaconazole, sertindole, sertralion, silicates, sildenafil, simvastatin, sitosterol, sotalol, spaglumic acid, sparfloxacin, spectinomycin, spiramycin, spirapril, spironolactone, stavudine, streptomycin, sucralfate, sufentanil, sulbactam, sulphonamides, sulfasalazine, sulpiride, sultamicillin, sultiam, sumatriptan, suxamethonium chloride, tacrine, tacrolimus, taliolol, tamoxifen, taurolidine, tazarotene, temazepam, teniposide, tenoxicam, terazosin, terbinafine, terbutaline, terfenadine, terlipressin, tertatolol, tetracyclins, teryzoline, theobromine, theophylline, butizine, thiamazole, phenothiazines, thiotepa, tiagabine, tiapride, propionic acid derivatives, ticlopidine, timolol, tinidazole, tioconazole, tioguanine, tioxolone, tiropramide, tizanidine, tolazoline, tolbutamide, tolcapone, tolnaftate, tolperisone, topotecan, torasemide, antioestrogens, tramadol, tramazoline, trandolapril, tranylcypromine, trapidil, trazodone, triamcinolone and triamcinolone derivatives, triamterene, trifluperidol, trifluridine, trimethoprim, trimipramine, tripelennamine, triprolidine, trifosfamide, tromantadine, trometamol, tropalpin, troxerutine, tulobuterol, tyramine, tyrothricin, urapidil, ursodeoxycholic acid, chenodeoxycholic acid, valaciclovir, valproic acid, vancomycin, vecuronium chloride, Viagra, venlafaxine, verapamil, vidarabine, vigabatrin, viloazine, vinblastine, vincamine, vincristine, vindesine, vinorelbine, vinpocetine, viquidil, warfarin, xantinol nicotinate, xipamide, zafirlukast, zalcitabine, zidovudine, zolmitriptan, zolpidem, zoplicone, zotipine and the like. See, e.g., U.S. Pat. No. 6,897,205; see also U.S. Pat. No. 6,838,528; U.S. Pat. No. 6,497,729.

Examples of therapeutic agents employed in conjunction with the invention include, rapamycin, 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin 40-O-(2-Hydroxy)ethoxycar-bonylmethyl-rapamycin, 40-O-(3-Hydroxyl)propyl-rapamycin, 40-O-(6-Hydroxyl)hexyl-rapamycin, 40-O-[2-(2-Hydroxyl)ethoxy]ethyl-rapamycin, 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin, 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxyl)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), and 42-[3-hydroxy-2-(hydroxymethyl)-2-methylpropanoate]rapamycin (temsirolimus).

The active ingredients may, if desired, also be used in the form of their pharmaceutically acceptable salts or derivatives (meaning salts which retain the biological effectiveness and properties of the compounds of this invention and which are not biologically or otherwise undesirable), and in the case of chiral active ingredients it is possible to employ both optically active isomers and racemates or mixtures of diastereoisomers.

“Stability” as used herein in refers to the stability of the drug in a polymer coating deposited on a substrate in its final product form (e.g., stability of the drug in a coated stent). The term stability will define 5% or less degradation of the drug in the final product form.

“Active biological agent” as used herein refers to a substance, originally produced by living organisms, that can be used to prevent or treat a disease (meaning any treatment of a disease in a mammal, including preventing the disease, i.e. causing the clinical symptoms of the disease not to develop; inhibiting the disease, i.e. arresting the development of clinical symptoms; and/or relieving the disease, i.e. causing the regression of clinical symptoms). It is possible that the active biological agents of the invention may also comprise two or more active biological agents or an active biological agent combined with a pharmaceutical agent, a stabilizing agent or chemical or biological entity. Although the active biological agent may have been originally produced by living organisms, those of the present invention may also have been synthetically prepared, or by methods combining biological isolation and synthetic modification. By way of a non-limiting example, a nucleic acid could be isolated form from a biological source, or prepared by traditional techniques, known to those skilled in the art of nucleic acid synthesis. Furthermore, the nucleic acid may be further modified to contain non-naturally occurring moieties. Non-limiting examples of active biological agents include peptides, proteins, enzymes, glycoproteins, nucleic acids (including deoxyribonucleotide or ribonucleotide polymers in either single or double stranded form, and unless otherwise limited, encompasses known analogues of natural nucleotides that hybridize to nucleic acids in a manner similar to naturally occurring nucleotides), antisense nucleic acids, fatty acids, antimicrobials, vitamins, hormones, steroids, lipids, polysaccharides, carbohydrates and the like. They further include, but are not limited to, antirestenotic agents, antidiabetics, analgesics, antiinflammatory agents, antirheumatics, antihypotensive agents, antihypertensive agents, psychoactive drugs, tranquillizers, antiemetics, muscle relaxants, glucocorticoids, agents for treating ulcerative colitis or Crohn's disease, antiallergics, antibiotics, antiepileptics, anticoagulants, antimycotics, antitussives, arteriosclerosis remedies, diuretics, proteins, peptides, enzymes, enzyme inhibitors, gout remedies, hormones and inhibitors thereof, cardiac glycosides, immunotherapeutic agents and cytokines, laxatives, lipid-lowering agents, migraine remedies, mineral products, otologicals, anti parkinson agents, thyroid therapeutic agents, spasmolytics, platelet aggregation inhibitors, vitamins, cytostatics and metastasis inhibitors, phytopharmaceuticals and chemotherapeutic agents. Preferably, the active biological agent is a peptide, protein or enzyme, including derivatives and analogs of natural peptides, proteins and enzymes.

“Activity” as used herein refers to the ability of a pharmaceutical or active biological agent to prevent or treat a disease (meaning any treatment of a disease in a mammal, including preventing the disease, i.e. causing the clinical symptoms of the disease not to develop; inhibiting the disease, i.e. arresting the development of clinical symptoms; and/or relieving the disease, i.e. causing the regression of clinical symptoms). Thus the activity of a pharmaceutical or active biological agent should be of therapeutic or prophylactic value.

“Secondary, tertiary and quaternary structure” as used herein are defined as follows. The active biological agents of the present invention will typically possess some degree of secondary, tertiary and/or quaternary structure, upon which the activity of the agent depends. As an illustrative, non-limiting example, proteins possess secondary, tertiary and quaternary structure. Secondary structure refers to the spatial arrangement of amino acid residues that are near one another in the linear sequence. The α-helix and the β-strand are elements of secondary structure. Tertiary structure refers to the spatial arrangement of amino acid residues that are far apart in the linear sequence and to the pattern of disulfide bonds. Proteins containing more than one polypeptide chain exhibit an additional level of structural organization. Each polypeptide chain in such a protein is called a subunit. Quaternary structure refers to the spatial arrangement of subunits and the nature of their contacts. For example hemoglobin consists of two a and two 3 chains. It is well known that protein function arises from its conformation or three dimensional arrangement of atoms (a stretched out polypeptide chain is devoid of activity). Thus one aspect of the present invention is to manipulate active biological agents, while being careful to maintain their conformation, so as not to lose their therapeutic activity.

“Polymer” as used herein, refers to a series of repeating monomeric units that have been cross-linked or polymerized. Any suitable polymer can be used to carry out the present invention. It is possible that the polymers of the invention may also comprise two, three, four or more different polymers. In some embodiments, of the invention only one polymer is used. In some preferred embodiments a combination of two polymers are used. Combinations of polymers can be in varying ratios, to provide coatings with differing properties. Those of skill in the art of polymer chemistry will be familiar with the different properties of polymeric compounds.

“Therapeutically desirable morphology” as used herein refers to the gross form and structure of the pharmaceutical agent, once deposited on the substrate, so as to provide for optimal conditions of ex vivo storage, in vivo preservation and/or in vivo release. Such optimal conditions may include, but are not limited to increased shelf life, increased in vivo stability, good biocompatibility, good bioavailability or modified release rates. Typically, for the present invention, the desired morphology of a pharmaceutical agent would be crystalline or semi-crystalline or amorphous, although this may vary widely depending on many factors including, but not limited to, the nature of the pharmaceutical agent, the disease to be treated/prevented, the intended storage conditions for the substrate prior to use or the location within the body of any biomedical implant. Preferably at least 10%, 20%, 30%, 40%, 50%, 60%, 70%, 80%, 90% or 100% of the pharmaceutical agent is in crystalline or semi-crystalline form.

“Stabilizing agent” as used herein refers to any substance that maintains or enhances the stability of the biological agent. Ideally these stabilizing agents are classified as Generally Regarded As Safe (GRAS) materials by the US Food and Drug Administration (FDA). Examples of stabilizing agents include, but are not limited to carrier proteins, such as albumin, gelatin, metals or inorganic salts. Pharmaceutically acceptable excipient that may be present can further be found in the relevant literature, for example in the Handbook of Pharmaceutical Additives: An International Guide to More Than 6000 Products by Trade Name, Chemical, Function, and Manufacturer; Michael and Irene Ash (Eds.); Gower Publishing Ltd.; Aldershot, Hampshire, England, 1995.

“Compressed fluid” as used herein refers to a fluid of appreciable density (e.g., >0.2 g/cc) that is a gas at standard temperature and pressure. “Supercritical fluid”, “near-critical fluid”, “near-supercritical fluid”, “critical fluid”, “densified fluid” or “densified gas” as used herein refers to a compressed fluid under conditions wherein the temperature is at least 80% of the critical temperature of the fluid and the pressure is at least 50% of the critical pressure of the fluid.

Examples of substances that demonstrate supercritical or near critical behavior suitable for the present invention include, but are not limited to carbon dioxide, isobutylene, ammonia, water, methanol, ethanol, ethane, propane, butane, pentane, dimethyl ether, xenon, sulfur hexafluoride, halogenated and partially halogenated materials such as chlorofluorocarbons, hydrochlorofluorocarbons, hydrofluorocarbons, perfluorocarbons (such as perfluoromethane and perfuoropropane, chloroform, trichloro-fluoromethane, dichloro-difluoromethane, dichloro-tetrafluoroethane) and mixtures thereof.

“Sintering” as used herein refers to the process by which parts of the matrix or the entire polymer matrix becomes continuous (e.g., formation of a continuous polymer film). As discussed below, the sintering process is controlled to produce a fully conformal continuous matrix (complete sintering) or to produce regions or domains of continuous coating while producing voids (discontinuities) in the matrix. As well, the sintering process is controlled such that some phase separation is obtained between polymer different polymers (e.g., polymers A and B) and/or to produce phase separation between discrete polymer particles. Through the sintering process, the adhesions properties of the coating are improved to reduce flaking of detachment of the coating from the substrate during manipulation in use. As described below, in some embodiments, the sintering process is controlled to provide incomplete sintering of the polymer matrix. In embodiments involving incomplete sintering, a polymer matrix is formed with continuous domains, and voids, gaps, cavities, pores, channels or, interstices that provide space for sequestering a therapeutic agent which is released under controlled conditions. Depending on the nature of the polymer, the size of polymer particles and/or other polymer properties, a compressed gas, a densified gas, a near critical fluid or a super-critical fluid may be employed. In one example, carbon dioxide is used to treat a substrate that has been coated with a polymer and a drug, using dry powder and RESS electrostatic coating processes. In another example, isobutylene is employed in the sintering process. In other examples a mixture of carbon dioxide and isobutylene is employed.

When an amorphous material is heated to a temperature above its glass transition temperature, or when a crystalline material is heated to a temperature above a phase transition temperature, the molecules comprising the material are more mobile, which in turn means that they are more active and thus more prone to reactions such as oxidation. However, when an amorphous material is maintained at a temperature below its glass transition temperature, its molecules are substantially immobilized and thus less prone to reactions. Likewise, when a crystalline material is maintained at a temperature below its phase transition temperature, its molecules are substantially immobilized and thus less prone to reactions. Accordingly, processing drug components at mild conditions, such as the deposition and sintering conditions described herein, minimizes cross-reactions and degradation of the drug component. One type of reaction that is minimized by the processes of the invention relates to the ability to avoid conventional solvents which in turn minimizes autoxidation of drug, whether in amorphous, semi-crystalline, or crystalline form, by reducing exposure thereof to free radicals, residual solvents and autoxidation initiators.

“Rapid Expansion of Supercritical Solutions” or “RESS” as used herein involves the dissolution of a polymer into a compressed fluid, typically a supercritical fluid, followed by rapid expansion into a chamber at lower pressure, typically near atmospheric conditions. The rapid expansion of the supercritical fluid solution through a small opening, with its accompanying decrease in density, reduces the dissolution capacity of the fluid and results in the nucleation and growth of polymer particles. The atmosphere of the chamber is maintained in an electrically neutral state by maintaining an isolating “cloud” of gas in the chamber. Carbon dioxide or other appropriate gas is employed to prevent electrical charge is transferred from the substrate to the surrounding environment.

“Bulk properties” properties of a coating including a pharmaceutical or a biological agent that can be enhanced through the methods of the invention include for example: adhesion, smoothness, conformality, thickness, and compositional mixing.

“Electrostatically charged” or “electrical potential” or “electrostatic capture” as used herein refers to the collection of the spray-produced particles upon a substrate that has a different electrostatic potential than the sprayed particles. Thus, the substrate is at an attractive electronic potential with respect to the particles exiting, which results in the capture of the particles upon the substrate. i.e. the substrate and particles are oppositely charged, and the particles transport through the fluid medium of the capture vessel onto the surface of the substrate is enhanced via electrostatic attraction. This may be achieved by charging the particles and grounding the substrate or conversely charging the substrate and grounding the particles, or by some other process, which would be easily envisaged by one of skill in the art of electrostatic capture.

Means for creating the bioabsorbable polymer(s)+drug (s) matrix on the stent-form—forming the final device:
Spray coat the stent-form with drug and polymer as is done in Micell process (e-RESS, e-DPC, compressed-gas sintering).
Perform multiple and sequential coating-sintering steps where different materials may be deposited in each step, thus creating a laminated structure with a multitude of thin layers of drug(s), polymer(s) or drug+polymer that build the final stent.
Perform the deposition of polymer(s)+drug(s) laminates with the inclusion of a mask on the inner (luminal) surface of the stent. Such a mask could be as simple as a non-conductive mandrel inserted through the internal diameter of the stent form. This masking could take place prior to any layers being added, or be purposefully inserted after several layers are deposited continuously around the entire stent-form.

Another advantage of the present invention is the ability to create a stent with a controlled (dialed-in) drug-elution profile. Via the ability to have different materials in each layer of the laminate structure and the ability to control the location of drug(s) independently in these layers, the method enables a stent that could release drugs at very specific elution profiles, programmed sequential and/or parallel elution profiles. Also, the present invention allows controlled elution of one drug without affecting the elution of a second drug (or different doses of the same drug).

The embodiments incorporating a stent form or framework provide the ability to radiographically monitor the stent in deployment. In an alternative embodiment, the inner-diameter of the stent can be masked (e.g. by a non-conductive mandrel). Such masking would prevent additional layers from being on the interior diameter (abluminal) surface of the stent. The resulting configuration may be desirable to provide preferential elution of the drug toward the vessel wall (luminal surface of the stent) where the therapeutic effect of anti-restenosis is desired, without providing the same antiproliferative drug(s) on the abluminal surface, where they may retard healing, which in turn is suspected to be a cause of late-stage safety problems with current DESs.

The present invention provides numerous advantages. The invention is advantageous allows for employing a platform combining layer formation methods based on compressed fluid technologies; electrostatic capture and sintering methods. The platform results in drug eluting stents having enhanced therapeutic and mechanical properties. The invention is particularly advantageous in that it employs optimized laminate polymer technology. In particular, the present invention allows the formation of discrete layers of specific drug platforms.

Conventional processes for spray coating stents require that drug and polymer be dissolved in solvent or mutual solvent before spray coating can occur. The platform provided herein the drugs and polymers are coated on the stent framework in discrete steps, which can be carried out simultaneously or alternately. This allows discrete deposition of the active agent (e.g.; a drug) within a polymer matrix thereby allowing the placement of more than one drug on a single medical device with or without an intervening polymer layer. For example, the present platform provides a dual drug eluting stent.

Some of the advantages provided by the subject invention include employing compressed fluids (e.g., supercritical fluids, for example E-RESS based methods); solvent free deposition methodology; a platform that allows processing at lower temperatures thereby preserving the qualities of the active agent and the polymer matrix; the ability to incorporate two, three or more drugs while minimizing deleterious effects from direct interactions between the various drugs and/or their excipients during the fabrication and/or storage of the drug eluting stents; a dry deposition; enhanced adhesion and mechanical properties of the layers on the stent framework; precision deposition and rapid batch processing; and ability to form intricate structures.

In one embodiment, the present invention provides a multi-drug delivery platform which produces strong, resilient and flexible drug eluting stents including an anti-restenosis drug (e.g.; a limus or taxol) and anti-thrombosis drug (e.g.; heparin or an analog thereof) and well characterized bioabsorbable polymers. The drug eluting stents provided herein minimize potential for thrombosis, in part, by reducing or totally eliminating thrombogenic polymers and reducing or totally eliminating residual drugs that could inhibit healing.

The platform provides optimized delivery of multiple drug therapies for example for early stage treatment (restenosis) and late-stage (thrombosis).

The platform also provides an adherent coating which enables access through tortuous lesions without the risk of the coating being compromised.

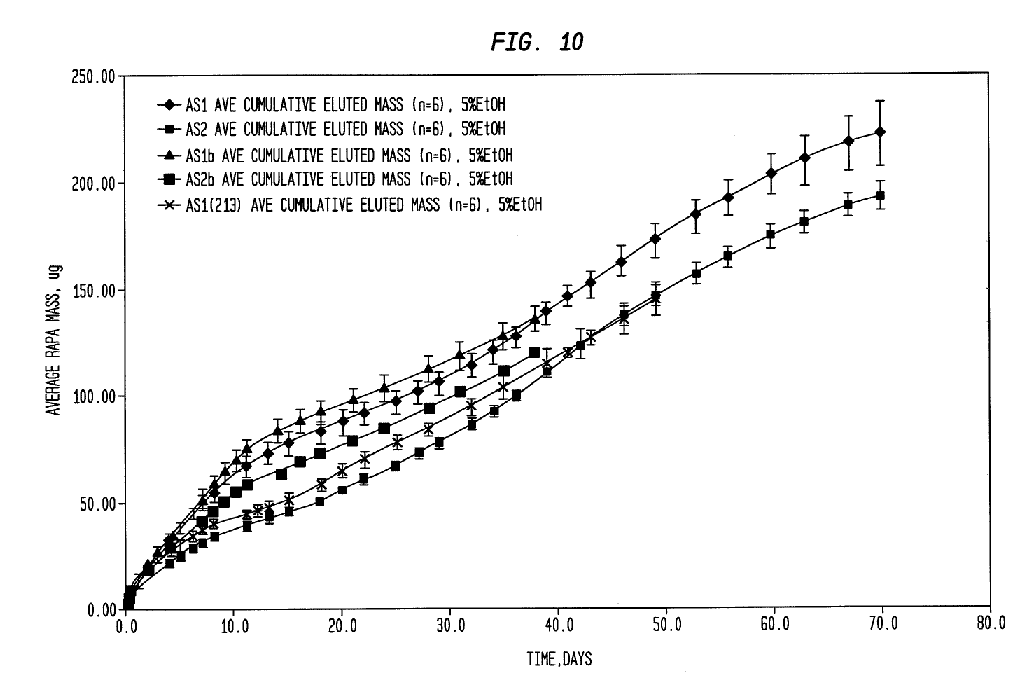

Another advantage of the present platform is the ability to provide highly desirable eluting profiles (e.g., the profile illustrated in FIGS. 10-12).

Advantages of the invention include the ability to reduce or completely eliminate potentially thrombogenic polymers as well as possibly residual drugs that may inhibit long term healing. As well, the invention provides advantageous stents having optimized strength and resilience if coatings which in turn allows access to complex lesions and reduces or completely eliminates delamination. Laminated layers of bioabsorbable polymers allow controlled elution of one or more drugs.

The platform provided herein reduces or completely eliminates shortcoming that have been associated with conventional drug eluting stents. For example, the platform provided herein allows for much better tuning of the period of time for the active agent to elute and the period of time necessary for the polymer matrix to resorb thereby minimizing thrombosis and other deleterious effects associate with poorly controlled drug release.

The present invention provides several advantages which overcome or attenuate the limitations of current technology for bioabsorbable stents. For example, an inherent limitation of conventional bioabsorbable polymeric materials relates to the difficulty in forming to a strong, flexible, deformable (e.g. balloon deployable) stent with low profile. The polymers generally lack the strength of high-performance metals. The present invention overcomes these limitations by creating a laminate structure in the essentially polymeric stent. Without wishing to be bound by any specific theory or analogy, the increased strength provided by the stents of the invention can be understood by comparing the strength of plywood vs. the strength of a thin sheet of wood.

Embodiments of the invention involving a thin metallic stent-framework provide advantages including the ability to overcome the inherent elasticity of most polymers. It is generally difficult to obtain a high rate (e.g., 100%) of plastic deformation in polymers (compared to elastic deformation where the materials have some ‘spring back’ to the original shape). Again, without wishing to be bound by any theory, the central metal stent framework (that would be too small and weak to serve as a stent itself) would act like wires inside of a plastic, deformable stent, basically overcoming any ‘elastic memory’ of the polymer.

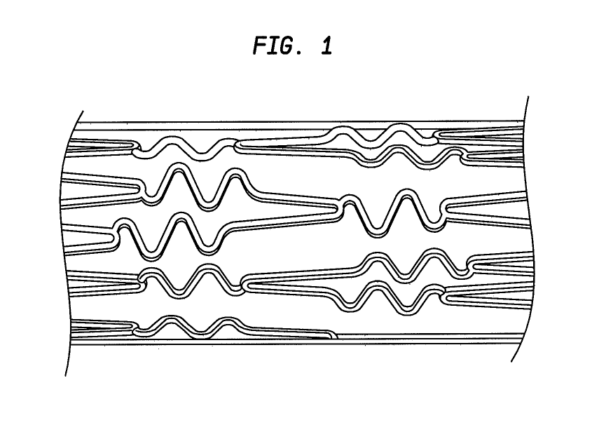

FIG. 1 depicts an image of an embodiment of a coated stent. The technology utilizes supercritical fluids and an E-RESS coating method that is solvent free, and conducted at low temperature. Multiple drugs may be coated using this method. The method of coating comprises a spraying dry components on a substrate, such that there is no bleeding of layers into each other. This method provides adhesion of layers and desirable mechanical properties. This method provides precision of layers and enables rapid batch processing.

The technology provided herein is capable of making novel devices. It enables laminate structures, and allows forming intricate, novel devices. The unique laminate structures provide structural control without introducing new materials or a new delivery system. The technology has been demonstrated for new drug eluting coatings and coated membranes, as shown herein.

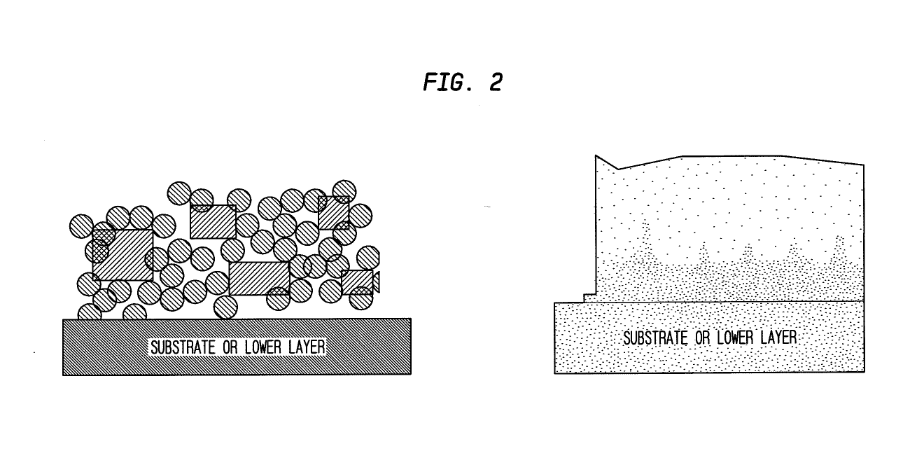

FIG. 2 depicts the process steps of an embodiment of coating a substrate. The process technology of this embodiment comprises a first step of electrostatic coating a substrate or lower layer. In this step, nano and microparticles of polymer(s) and drug(s) are electrostatically captured, dry upon a substrate such as a stent or stent form. The second step may comprise sintering wherein polymer nanoparticles are fused via supercritical fluid (SCF), using no solvents or high temperatures. The final material provides a smooth adherently laminated layer with precise control over location of drug(s).

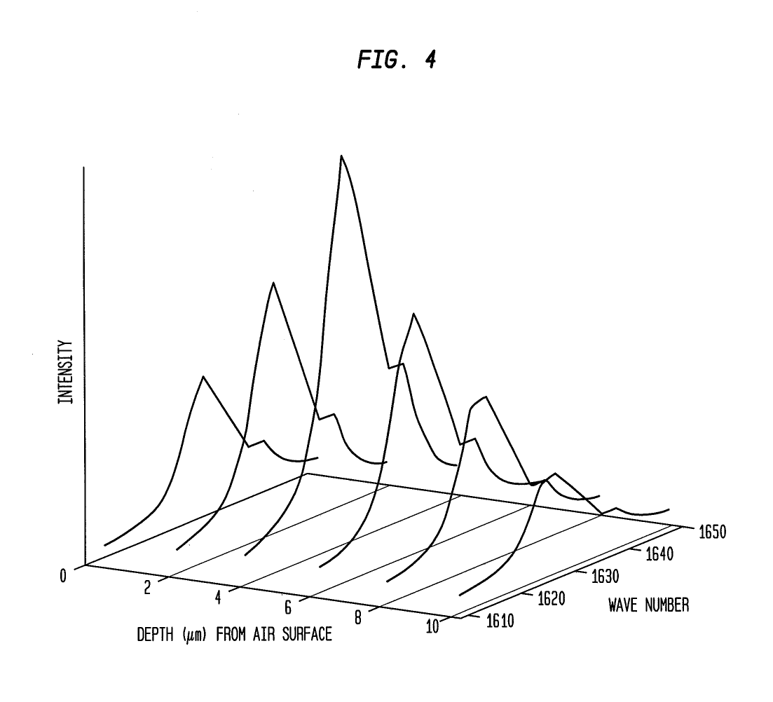

FIG. 4 depicts the ability of the coatings of the present methods and devices produced thereby to load drug at intended locations in the device. The drug distribution may be controlled using the methods as provided herein through the thickness of the coating and drug may be controlled in the coating such that it is evident in the surface of the coating. For example, FIG. 4 shows that the drug may be loaded purposefully in the center of a 10 micron coating produced using methods described herein, tested by Confocal Raman spectra testing, wherein the drug peak is at about 1620 (wave number).

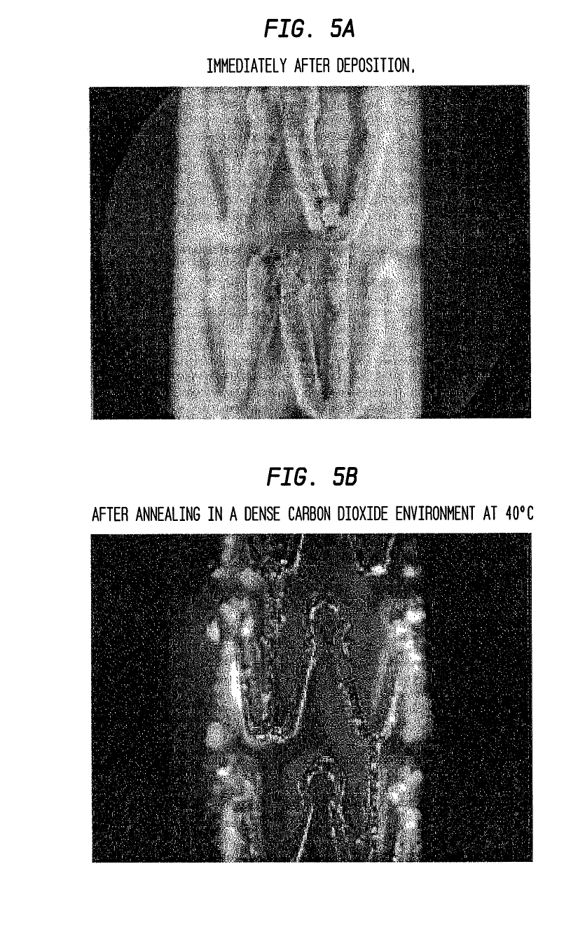

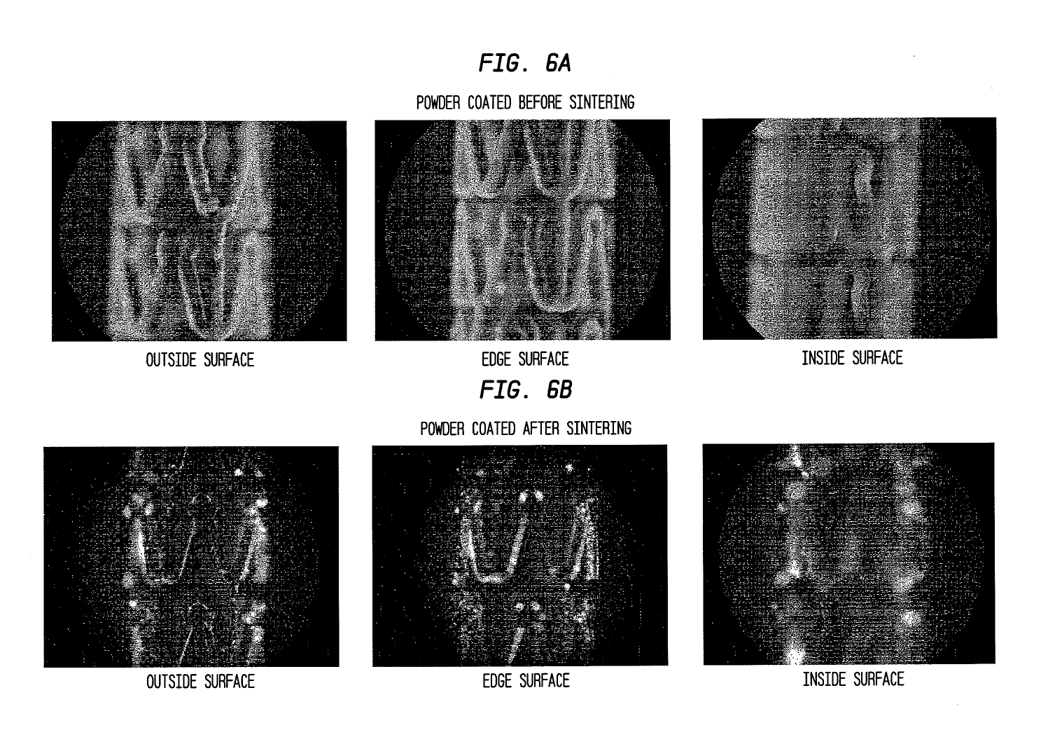

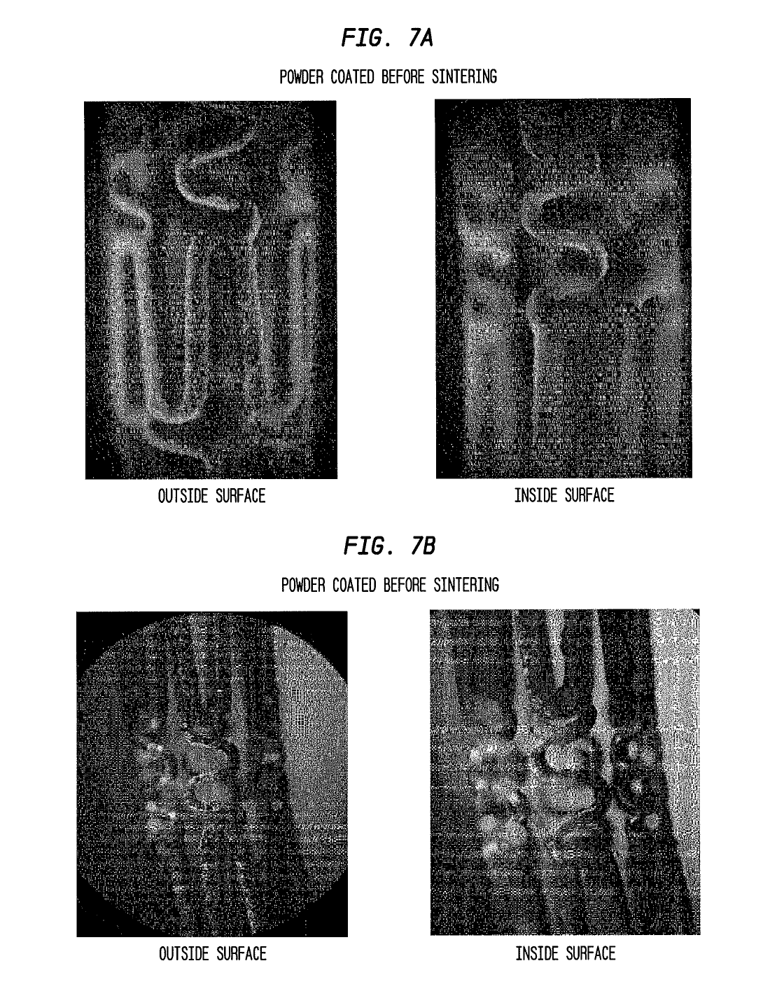

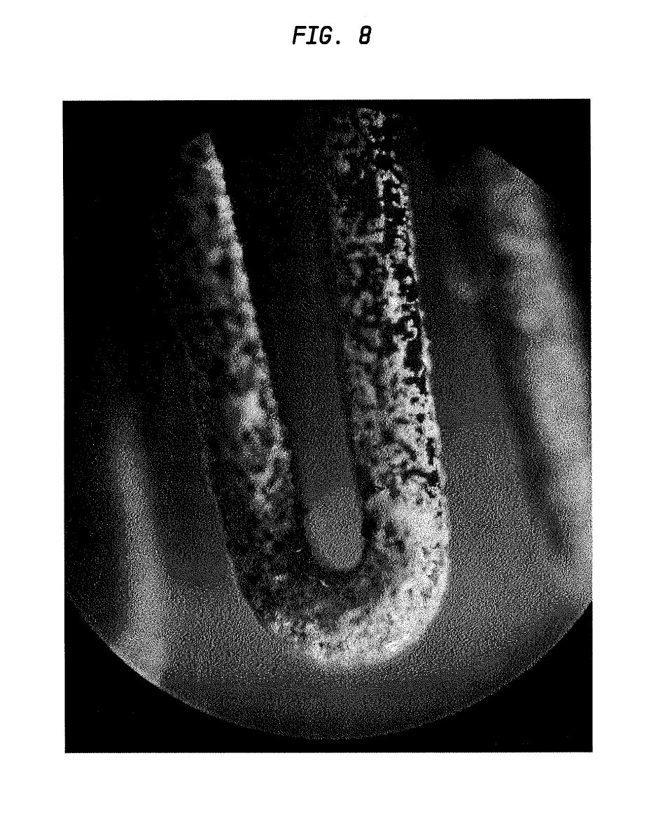

FIG. 5 shows a Drug-Polymer coated coronary stent (a) immediately after deposition, (b) after annealing in a dense carbon dioxide environment at 40° C. The stents produced in example 1 were examined by optical microscopy, at 40× magnification with back and side lighting. This method was used to provide a coarse qualitative representation of coating uniformity and to generally demonstrate the utility of the low-temperature CO2 annealing step. The resulting photos shown in FIG. 5, demonstrate the differences in appearance (a) before and (b) after annealing in dense carbon dioxide at 40 C. Photos of the outside, edge and inside surfaces are presented in FIG. 6(a), prior to sintering, which clearly shows nanoparticle deposition equally on all surfaces of the stent, and FIG. 6(b) after sintering, with the film showing a smooth and optically transparent polymer. FIG. 7 shows additional 40× magnified images of Rapamycin/PEVA/PBMA coated stents, showing the outside and inside surfaces, (a) before sintering, further demonstrating the nanoparticle deposition equally on all surfaces of the stent and (b) after sintering, showing a smooth and optically transparent polymer film. FIG. 8 shows a 100× magnified mages of Rapamycin/PEVA/PBMA Coated Stents. Crystalline drug is clearly visible embedded within a highly uniform polymer coating.

### EXAMPLES

The following examples are given to enable those skilled in the art to more clearly understand and to practice the present invention. They should not be considered as limiting the scope of the invention, but merely as being illustrative and representative thereof.

### Example 1

### Coating of Stents with Rapamycin, Polyethylene-Co-Vinyl Acetate (PEVA) and Polybutyl Methacrylate (PBMA)

Micronized Rapamycin was purchased from LC Laboratories. PBMA (Mw=.about.237 k) and PEVA (33% vinyl acetate content) were purchased from Aldrich Chemicals. Two kinds of stents were used: 3 mm TriStar® from Guidant and 6 cell.times.8-mm, BX Velocity® from Cordis. The stents were coated by dry electrostatic capture followed by supercritical fluid sintering, using 3 stents/coating run and 3 runs/data set. The coating apparatus is as described herein. Analysis of the coated stents was performed by multiple techniques on both stents and coupons with relevant control experiments.

In this example a 1:1 ratio of PEVA and PBMA is dissolved in a Dichlorofluoromethane (CC1.sub.2FH), which is a compressed gas solvent known to be in the class of “Freon” chemicals. The physical properties of this particular Freon are as follows:

### BP=8.9 C

### Tc=178.33 C

Pc=751.47 psig Dc=0.526014 g/cc

A solution was formed by mixing 30 mg of the combined polymers per gram dichlorofluoromethane. The solution was then maintained at 60.degree. C. at vapor pressure (approx 28 psig) until the solution was ready to spray. The solution was then pressurized by adding an immiscible gas to the top of the vessel—typically Helium. Adding Helium compressed the Freon+polymer solution up to 700 (+/−50 psig), which resulted in a compressed fluid. The polymer+Freon solution was then pushed through a nozzle having an inner diameter of 0.005″ by continuous addition of Helium into the vessel. The solvent (dichlorofluoromethane) is rapidly vaporized coming out of the nozzle (which is heated to 120 C), as it's boiling point is significantly below room temperature. The Drug is deposited by dry powder spray coating. Between 10-30 mg of drug are charged into a small volume of tubing, which is then pressurized with gaseous carbon dioxide to 400 psig. The mixture flows through a nozzle having an inner diameter of 0.187″ into the coating vessel where the stents are held. During electrostatic deposition, the stent is charged and the nozzles are grounded.

### Example 2

### Scanning Electron Microscopy Analysis of Rapamycin/PEVA/PBM Coated Stents

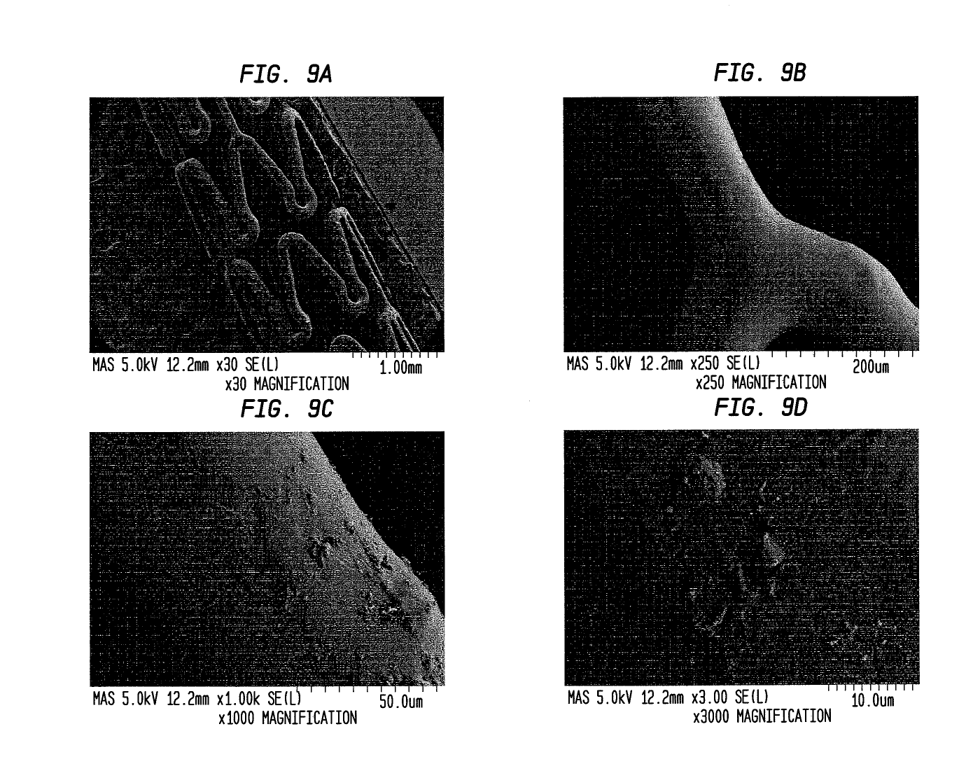

The stents produced in Example 1 were examined by scanning electron microscopy, and the resulting images presented in FIG. 9 at (a) ×30 magnification, (b) ×250 magnification, (c) ×1000 magnification and (d) ×3000 magnification. Clearly the nanoparticles have been sintered to an even and conformal film, with a surface topology of less than 5 microns, and demonstrate clear evidence of embedded crystalline rapamycin.

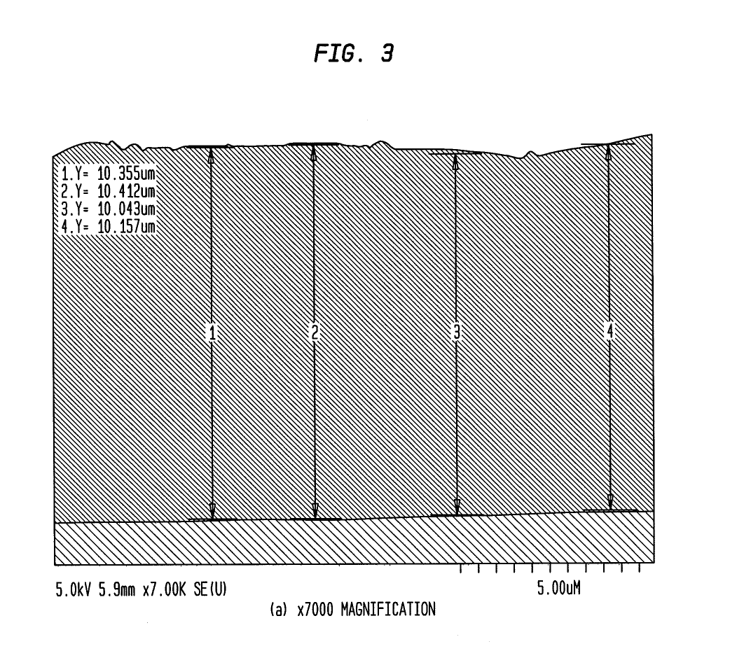

Cross-sectional (FIB) images were also acquired and are shown in FIG. 3 at 7000×. An even coating, 101, of consistent thickness is visible. Four cross-sectional thicknesses were measured: (1) 10.355 μM, (2) 10.412 μM, (3) 10.043 μM and (4) 10.157 μM, to give an average thickness of 10.242 μM, with only 2% (±0.2 μM) variation. Thus, FIG. 3 depicts a coating thickness of an embodiment coated device made according to a method described herein. In this embodiment device, the mean coating thickness is 10.2+/−0.2 microns, and the image shows a thin, conformal, defect-free coating at a target thickness.

### Example 3

In this example illustrates embodiments that provide a coated coronary stent, comprising: a stent framework and a rapamycin-polymer coating wherein at least part of rapamycin is in crystalline form and the rapamycin-polymer coating comprises one or more resorbable polymers.

In these experiments two different polymers were employed:

Polymer A:—50:50 PLGA-Ester End Group, MW˜90 kD, degradation rate ˜70 days

Polymer B:—50:50 PLGA-Carboxylate End Group, MW˜29 kD, degradation rate ˜28 days

Metal stents were coated as follows:

AS1: Polymer A/Rapamycin/Polymer A/Rapamycin/Polymer A

AS2: Polymer A/Rapamycin/Polymer A/Rapamycin/Polymer B

AS1 (B): Polymer B/Rapamycin/Polymer B/Rapamycin/Polymer B

AS1b: Polymer A/Rapamycin/Polymer A/Rapamycin/Polymer A

AS2b: Polymer A/Rapamycin/Polymer A/Rapamycin/Polymer B

Elution results are illustrated in FIGS. 10-12.

The in vitro pharmaceutical agent elution profile may be determined by a procedure comprising contacting the device with an elution media comprising ethanol (5%) wherein the pH of the media is about 7.4 and wherein the device is contacted with the elution media at a temperature of about 37° C. The elution media containing the device is optionally agitating the elution media during the contacting step. The device is removed (and/or the elution media is removed) at least at designated time points (e.g. 1 h, 3 h, 5 h, 7 h, 1 d or 24 hrs, and daily up to 28 d) (e.g. 1 week, 2 weeks, and 10 weeks). The elution media is then assayed for determination of the pharmaceutical agent content. The elution media is replaced at each time point with fresh elution media to avoid saturation of the elution media. Calibration standards containing known amounts of drug were also held in elution media for the same durations as the samples and used at each time point to determine the amount of drug eluted at that time (in absolute amount and as a cumulative amount eluted).

In this Example, as noted above, devices were coated tested using this method. In these experiments two different polymers were employed: Polymer A:—50:50 PLGA-Ester End Group, weight average MW˜90 kD, degradation rate ˜70 days; Polymer B:—50:50 PLGA-Carboxylate End Group, weight average MW˜29 kD, degradation rate ˜28 days. Metal stents were coated as follows:

AS1: (n=6) Polymer A/Rapamycin/Polymer A/Rapamycin/Polymer A;

AS2: (n=6) Polymer A/Rapamycin/Polymer A/Rapamycin/Polymer B;

AS1 (213): (n=6) Polymer B/Rapamycin/Polymer B/Rapamycin/Polymer B;

AS1b: (n=6) Polymer A/Rapamycin/Polymer A/Rapamycin/Polymer A;

AS2b: (n=6) Polymer A/Rapamycin/Polymer A/Rapamycin/Polymer B.

The in vitro pharmaceutical agent elution profile was determined by contacting each device with an elution media comprising ethanol (5%) wherein the pH of the media is about 7.4 and wherein the device was contacted with the elution media at a temperature of about 37° C. The elution media was removed from device contact at least at 1 h, 3 h, 5 h, 7 h, 1 d, and at additional time points up to 70 days (See FIGS. 10-12). The elution media was then assayed for determination of the pharmaceutical agent content (in absolute amount and cumulative amount eluted). The elution media was replaced at each time point with fresh elution media to avoid saturation of the elution media. Calibration standards containing known amounts of drug were also held in elution media for the same durations as the samples and assayed at each time point to determine the amount of drug eluted at that time (in absolute amount and as a cumulative amount eluted), compared to a blank comprising Spectroscopic grade ethanol (95%). Elution profiles as shown in FIGS. 10-12, showing the average amount of rapamycin eluted at each time point (average of all stents tested) in micrograms. The first column of Table 2 shows for each coating (AS1, AS2, AS(213), AS1b, AS2b), the average amount of rapamycin in ug loaded on the stents (average of n=6 stents for each coating). The second column of Table 2 shows for each coating (AS2, AS(213), AS1b, AS2b), the average amount of polymer in ug loaded on the stents (average of n=6 stents for each coating). The third column of Table 2 shows the total amount of rapamycin and polymer in ug loaded on the stents (column 1+column 2).

TABLE 2

| | | | Ave. |
|---|---|---|---|
| Stent | Ave. Rapa, | Ave. Poly, | Total |
| Coating | ug | ug | Mass, ug |
| AS1 | 175 | | |
| AS2 | 153 | 717 | 870 |
| AS1(213) | 224 | 737 | 961 |
| AS1b | 171 | 322 | 493 |
| AS2b | 167 | 380 | 547 |

FIG. 10: Rapamycin Elution Profile of coated stents (PLGA/Rapamycin coatings) where the elution profile was determined by static elution media of 5% EtOH/water, pH 7.4, 37° C. FIG. 10 depicts AS1 and AS2 as having statistically different elution profiles; AS2 and AS2b have statistically different profiles (where AS2b has about half of the polymer of AS2); AS1 and AS1b are not statistically different (where AS1b has about half of the polymer of AS1); and AS2 and AS1(213) begin to converge at 35 days (where these have about the same amount of polymer, but AS1(213) has only Polymer B, whereas AS2 has Polymer A and Polymer B, and AS1(213) has more rapamycin loaded on average than AS2). FIG. 10 suggests that the coating thickness does not affect elution rates from 3095 polymer (Polymer A), but does affect elution rates from the 213 polymer (Polymer B).

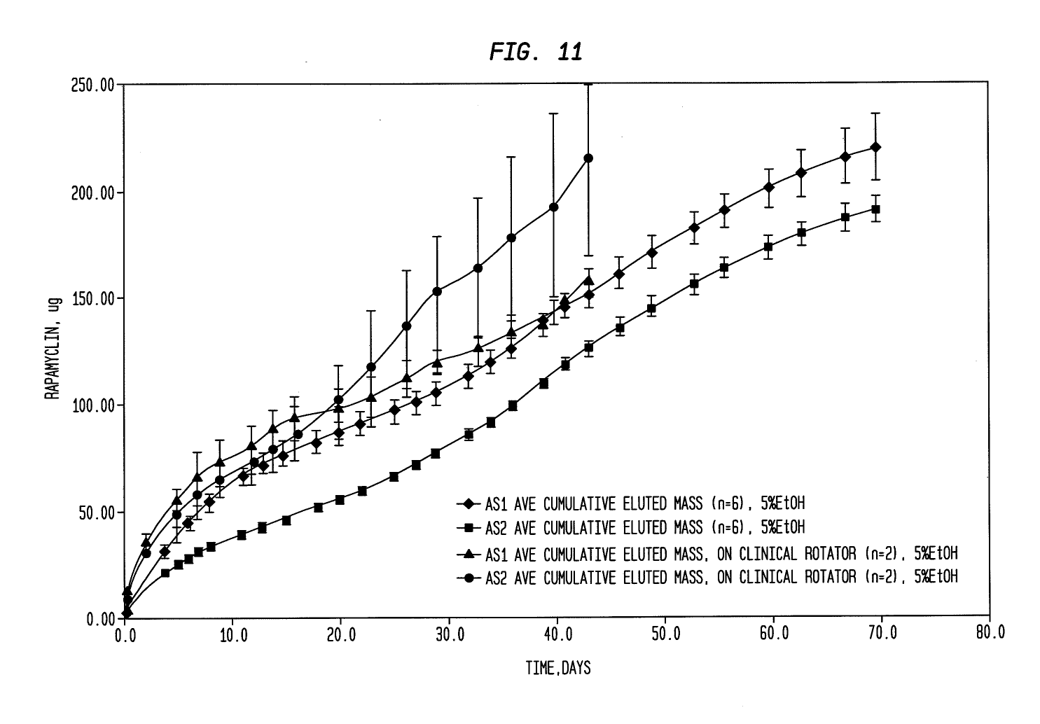

FIG. 11: Rapamycin Elution Rates of coated stents (PLGA/Rapamycin coatings) where the static elution profile was compared with agitated elution profile by an elution media of 5% EtOH/water, pH 7.4, 37° C. FIG. 11 depicts that agitation in elution media increases the rate of elution for AS2 stents, but is not statistically significantly different for AS1 stents. The profiles are based on two stent samples.

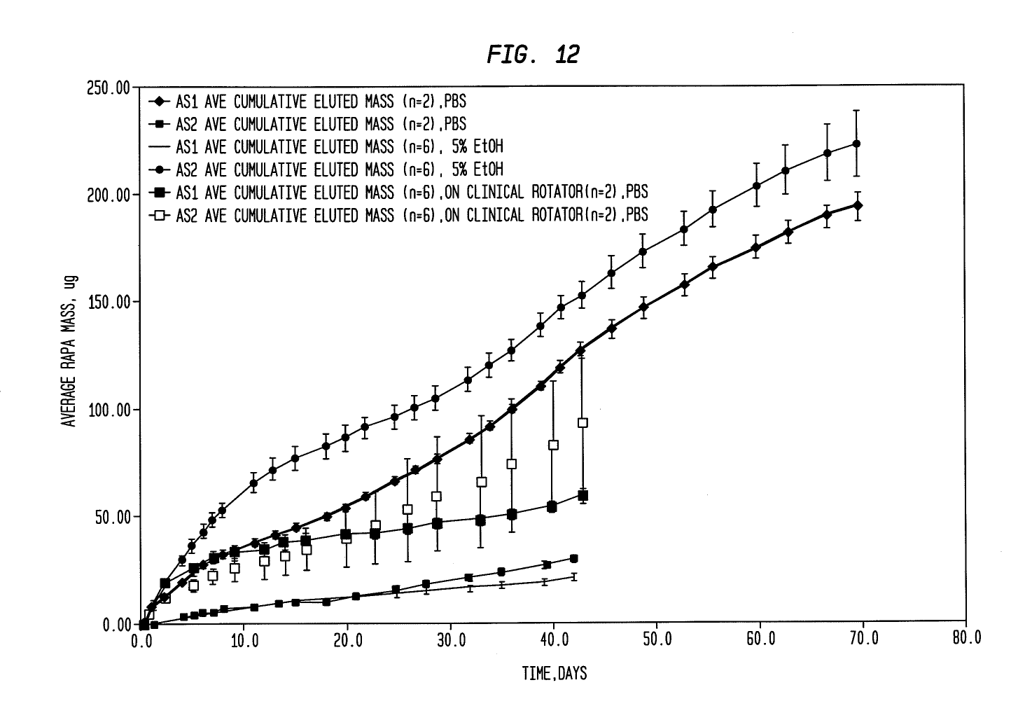

FIG. 12 Rapamycin Elution Profile of coated stents (PLGA/Rapamycin coatings) where the elution profile by 5% EtOH/water, pH 7.4, 37° C. elution buffer was compare with the elution profile using phosphate buffer saline pH 7.4, 37° C. FIG. 12 depicts that agitating the stent in elution media increases the elution rate in phosphate buffered saline, but the error is much greater.

In some embodiments, the stent elutes at least 50 micrograms of the rapamycin in vitro at day 10.

In some embodiments, the PLGA comprises an ester end group.

In some embodiments, the elution media is static during elution. In some embodiments, the elution media is agitated during elution.

In some embodiments, the stent elutes at most 60 micrograms of the rapamycin in vitro at day 10 when the elution media is static during elution.

In some embodiments, the stent elutes at least 25 micrograms of the rapamycin in vitro at day 10.

Provided herein is a stent comprising a stent framework; a plurality of layers deposited on said stent framework to form said coronary stent; wherein at least one of said layers comprises a 50:50 PLGA bioabsorbable polymer and at least one of said layers comprises rapamycin; wherein at least part of rapamycin is in crystalline form, wherein the stent elutes at most 50% of the rapamycin in vitro at day 10 after the stent is contacted with an elution media comprising ethanol (5%) wherein a pH of the media is about 7.4 and wherein the elution media is at a temperature of about 37° C. Such a device can be found in FIGS. 10 to 12, at least, and as described in Example 3.

In some embodiments, the stent elutes at least 30% of the rapamycin in vitro at day 10.

In some embodiments, the PLGA comprises an ester end group.

In some embodiments, the elution media is static during elution. In some embodiments, the elution media is agitated during elution.

In some embodiments, the stent elutes at most 40% of the rapamycin in vitro at day 10 when the elution media is static during elution.

In some embodiments, the stent elutes at least 10% of the rapamycin in vitro at day 10.

In some embodiments, the stent elutes at least 50 micrograms of the rapamycin in vitro at day 10.

In some embodiments, the PLGA comprises an ester end group.

In some embodiments, the elution media is static during elution. In some embodiments, the elution media is agitated during elution.

In some embodiments, the stent elutes at most 60 micrograms of the rapamycin in vitro at day 10 when the elution media is static during elution.

In some embodiments, the stent elutes at least 25 micrograms of the rapamycin in vitro at day 10.

Provided herein is a method of preparing a stent comprising: providing a stent framework; depositing a coating on said stent framework to form said coronary stent; wherein said coating comprises rapamycin and a polymer wherein at least part of the rapamycin is in crystalline form and the polymer comprises PLGA, wherein depositing said coating comprises depositing polymer particles on said framework in dry powder form and depositing rapamycin particles on said framework in dry powder form; and sintering said coating under conditions that do not substantially modify the morphology of the rapamycin, wherein the stent elutes at most 50% of the rapamycin in vitro at day 10 after the stent is contacted with an elution media comprising ethanol (5%) wherein a pH of the media is about 7.4 and wherein the elution media is at a temperature of about 37° C. Such a method can be found described and tested as in FIGS. 10 to 12, at least and in Example 3.

In some embodiments, the stent elutes at least 30% of the rapamycin in vitro at day 10.

In some embodiments, the elution media is static during elution. In some embodiments, the elution media is agitated during elution.

In some embodiments, the stent elutes at most 40% of the rapamycin in vitro at day 10 when the elution media is static during elution.

In some embodiments, the PLGA comprises an ester end group.

In some embodiments, the stent elutes at least 10% of the rapamycin in vitro at day 10.

Provided herein is a stent comprising a stent framework; a plurality of layers deposited on said stent framework to form said coronary stent; wherein at least one of said layers comprises a 50:50 PLGA bioabsorbable polymer and at least one of said layers comprises a macrolide immunosuppressive drug; wherein at least part of macrolide immunosuppressive drug is in crystalline form, wherein the stent elutes at most 50% of the macrolide immunosuppressive drug in vitro at day 10 after the stent is contacted with an elution media comprising ethanol (5%) wherein a pH of the media is about 7.4 and wherein the elution media is at a temperature of about 37° C. Such a device can be found in FIGS. 10 to 12.

In some embodiments, the macrolide immunosuppressive drug comprises one or more of rapamycin, 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin, 40-O-(2-Hydroxy)ethoxycar-bonylmethyl-rapamycin, 40-O-(3-Hydroxyl)propyl-rapamycin, 40-O-(6-Hydroxyl)hexyl-rapamycin, 40-O-[2-(2-Hydroxyl)ethoxy]ethyl-rapamycin, 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin, 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxyl)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), and 42-[3-hydroxy-2-(hydroxymethyl)-2-methylpropanoate]rapamycin (temsirolimus).

In some embodiments, the macrolide immunosuppressive drug is at least 50% crystalline. In some embodiments, the rapamycin and polymer are in a same layer; in separate layers or form overlapping layers. In some embodiments, the coating comprises five layers deposited as follows: a first polymer layer, a first rapamycin layer, a second polymer layer, a second rapamycin layer and a third polymer layer.

In some embodiments, at least 50% of said rapamycin in powder form is crystalline or semicrystalline. In some embodiments, said rapamycin is at least 90% crystalline.

In some embodiments, said polymer is a mixture of two or more polymers. In some embodiments, said mixture of polymers forms a continuous film around particles of rapamycin.

The foregoing is illustrative of the present invention, and is not to be construed as limiting thereof. While embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of preparing a coronary stent comprising:
a. providing a stent framework;
b. depositing a plurality of layers on said stent framework to form said coronary stent; wherein at least one of said layers comprises a drug-polymer coating wherein at least part of the drug is in crystalline form and the polymer is a bioabsorbable polymer.

2. The method of claim 1, wherein the drug and polymer are in the same layer; in separate layers or in overlapping layers.

3. The method of claim 1, wherein the stent framework is made of a material selected form the group consisting of stainless steel, a metal alloy, and a cobalt chromium alloy.

4. The method of claim 1 wherein the stent framework is formed from a material comprising the following percentages by weight: 0.05-0.15 C, 1.00-2.00 Mn, 0.040 Si, 0.030 P, 0.3 S, 19.00-21.00 Cr, 9.00-11.00 Ni, 14.00-16.00 W, 3.00 Fe, and Bal. Co.

5. The method of claim 1, wherein the stent framework has a thickness of about 50% or less of a thickness of the coronary stent.

6. The method of claim 1, wherein the stent framework has a thickness of about 100 m or less.

7. The method of claim 1, wherein said bioabsorbable polymer is selected from the group consisting of PGA poly(glycolide), LPLA poly(1-lactide), DLPLA poly(dl-lactide), PCL poly(e-caprolactone) PDO, poly(dioxolane) PGA-TMC, 85/15 DLPLG p(dl-lactide-co-glycolide), 75/25 DLPL, 65/35 DLPLG, 50/50 DLPLG, TMC poly(trimethylcarbonate), p(CPP:SA) poly(1,3-bis-p-(carboxyphenoxy)propane-co-sebacic acid).

8. The method of claim 1 comprising depositing 4 or more layers.

9. The method of claim 1 wherein said layers comprise alternate drug and polymer layers.

10. The method of claim 9, wherein the drug layers are substantially free of polymer and the polymer layers are substantially free of drug.

11. The method of claim 10, wherein said one or more active agents comprise a macrolide immunosuppressive (limus) drug.

12. The method of claim 11, wherein the macrolide immunosuppressive drug comprises one or more of rapamycin, 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4,5′-Dihydroxypent-2′-en1′-yl)-rapamycin40-O-(2-Hydroxy)ethoxycar-bonylmethyl-rapamycin, 40-O-(3-Hydroxyl)propyl-rapamycin 40-O-(6-Hydroxyl)hexyl-rapamycin 40-O-[2-(2-Hydroxyl)ethoxy]ethyl-rapamycin 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin 40-O-(2-Nicotinoyloxy)ethyl-rapamycin, 40-O-[2-(N-Morpholino)acetoxy]ethyl-rapamycin 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxyl)ethyl-rapamycin, 28-O-Methyl-rapamycin, 40-O-(2-Aminoethyl)-rapamycin, 40-O-(2-Acetaminoethyl)-rapamycin 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), and 42-[3-hydroxy-2-(hydroxymethyl)-2-methylpropanoate]rapamycin (temsirolimus).

13. The method of claim 11, wherein said macrolide immunosuppressive drug is at least 50% crystalline.

14. The method of claim 11, wherein depositing a plurality of layers on said stent framework to form said coronary stent comprises depositing polymer particles on said framework by an RESS process.

15. The method of claim 1, wherein depositing a plurality of layers on said stent framework to form said coronary stent comprises depositing polymer particles on said framework in dry powder form.

16. A laminate coronary stent comprising
a. a stent framework;
b. a plurality of layers deposited on said stent framework to form said coronary stent; wherein at least one of said layers comprises a bioabsorbable polymer and at least one of said layers comprises one or more active agents; wherein at least part of the active agent is in crystalline form.

17. The stent of claim 16, wherein the active agent and polymer are in the same layer; in separate layers or form overlapping layers.

18. A coronary stent comprising
a. a stent framework;
b. a plurality of layers deposited on said stent framework to form said coronary stent; wherein at least one of said layers comprises a PLGA bioabsorbable polymer and at least one of said layers comprises rapamycin; wherein at least part of rapamycin is in crystalline form,

19. The coronary stent of claim 18 wherein the plurality of layers comprise five layers deposited as follows: a first polymer layer, a first rapamycin layer, a second polymer layer, a second rapamycin layer and a third polymer layer.

20. The stent of claim 18 wherein the stent framework is formed from a material comprising the following percentages by weight: 0.05-0.15 C, 1.00-2.00 Mn, 0.040 Si, 0.030 P, 0.3 S, 19.00-21.00 Cr, 9.00-11.00 Ni, 14.00-16.00 W, 3.00 Fe, and Bal. Co.

21. A method of preparing a coronary stent comprising:
a. providing a stent framework;
b. depositing a plurality of layers on said stent framework to form said coronary stent; wherein at least one of said layers comprises a bioabsorbable polymer; wherein depositing each layer of said plurality of layers on said stent framework comprises the following steps:
discharging at least one pharmaceutical agent and/or at least one active biological agent in dry powder form through a first orifice;
discharging the at least one polymer in dry powder form through said first orifice or through a second orifice;
depositing the polymer and pharmaceutical agent and/or active biological agent particles onto said framework, wherein an electrical potential is maintained between the framework and the polymer and pharmaceutical agent and/or active biological agent particles, thereby forming said layer; and
sintering said layer under conditions that do not substantially modify the morphology of said pharmaceutical agent and/or the activity of said biological agent.

22. A method of preparing a coronary stent comprising:
a. providing a stent framework;
b. depositing a plurality of layers on said stent framework to form said coronary stent; wherein at least one of said layers comprises a bioabsorbable polymer; at least one pharmaceutical agent in a therapeutically desirable morphology and/or at least one active biological agent; wherein depositing each layer of said plurality of layers on said stent framework comprises the following steps:
i. discharging the at least one pharmaceutical agent and/or at least one active biological agent in dry powder form through a first orifice;
ii. forming a supercritical or near supercritical fluid solution comprising at least one supercritical fluid solvent and at least one polymer and discharging said supercritical or near supercritical fluid solution through a second orifice under conditions sufficient to form solid particles of the polymer;
iii. depositing the polymer and pharmaceutical agent and/or active biological agent particles onto said framework, wherein an electrical potential is maintained between the framework and the polymer and pharmaceutical agent and/or active biological agent particles, thereby forming said layer; and
iv. sintering said layer under conditions that do not substantially modify the morphology of said pharmaceutical agent and/or the activity of said biological agent.

23. A method of preparing a coronary stent comprising:
a. providing a stent framework;
b. depositing a plurality of layers on said stent framework to form said coronary stent; wherein at least one of said layers comprises a bioabsorbable polymer; at least one pharmaceutical agent in a therapeutically desirable morphology and/or at least one active biological agent; wherein depositing each layer of said plurality of layers on said stent framework comprises the following steps:
i. forming a supercritical or near supercritical fluid solution comprising at least one supercritical fluid solvent and one or more pharmaceutical agents and/or at least one active biological agent discharging said supercritical or near supercritical fluid solution through a first orifice under conditions sufficient to form solid particles of said one or more pharmaceutical agents and/or at least one active biological agent;
ii. forming a supercritical or near supercritical fluid solution comprising at least one supercritical fluid solvent and at least one polymer and discharging said supercritical or near supercritical fluid solution through said first orifice or through a second orifice under conditions sufficient to form solid particles of the polymer;
iii. depositing the polymer and pharmaceutical agent and/or active biological agent particles onto said framework, wherein an electrical potential is maintained between the framework and the polymer and pharmaceutical agent and/or active biological agent particles, thereby forming said layer; and
iv. sintering said layer under conditions that do not substantially modify the morphology of said pharmaceutical agent and/or the activity of said biological agent.

24. The method of claim 21, further comprising discharging a third dry powder comprising a second pharmaceutical agent in a therapeutically desirable morphology in dry powder form and/or active biological agent whereby a layer comprising at least two different pharmaceutical agents and/or active biological agents is deposited on said framework or at least two layers each comprising one of two different pharmaceutical agents and/or active biological agents are deposited on said framework.

25. The method of claim 21, wherein the therapeutically desirable morphology of said pharmaceutical agent is crystalline or semi-crystalline.

26. The method of claim 21, wherein at least 50% of said pharmaceutical agent in powder form is crystalline or semi-crystalline.

27. The method of claim 21, wherein said bioabsorbable polymer is selected from PGA poly(glycolide), LPLA poly(1-lactide), DLPLA poly(dl-lactide), PCL poly(e-caprolactone) PDO, poly(dioxolane) PGA-TMC, 85/15 DLPLG p(dl-lactide-co-glycolide), 75/25 DLPL, 65/35 DLPLG, 50/50 DLPLG, TMC poly(trimethylcarbonate), p(CPP:SA) poly(1,3-bis-p-(carboxyphenoxy)propane-co-sebacic acid).

28. The method of claim 21 comprising depositing 4 or more layers.

29. The method of claim 21 wherein said layers comprise alternate drug and polymer layers.

30. The method of claim 29, wherein the drug layers are substantially free of polymer and the polymer layers are substantially free of drug.

31. The method of claim 21, wherein said one or more active agents comprise a macrolide immunosuppressive (limus) drug.

32. The method of claim 31, wherein the macrolide immunosuppressive drug comprises one or more of rapamycin, 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin 40-O-(2-Hydroxy)ethoxycar-bonylmethyl-rapamycin, 40-O-(3-Hydroxyl)propyl-rapamycin 40-O-(6-Hydroxyl)hexyl-rapamycin40-O-[2-(2-Hydroxyl)ethoxy]ethyl-rapamycin40-O-[(3S)-2,2-Dimethyldioxolan-3-yl]methyl-rapamycin, 40-O-[(2S)-2,3-Dihydroxyprop-1-yl]-rapamycin, 40-O(2-Acetoxy)ethyl-rapamycin 40-O-(2-Nicotinoyloxy)ethyl-rapamycin, 40-O-[2-(N-Morpholino)acetoxy]ethyl-rapamycin 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxyl)ethyl-rapamycin, 28-O-Methyl-rapamycin, 40-O-(2-Aminoethyl)-rapamycin, 40-O-(2-Acetaminoethyl)-rapamycin 40-25 0-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), and 42-[3-hydroxy-2-(hydroxymethyl)-2-methylpropanoate]rapamycin (temsirolimus).

33. A coated coronary stent, comprising:
a stent framework; and
a rapamycin-polymer coating wherein at least part of rapamycin is in crystalline form and the rapamycin-polymer coating comprises one or more resorbable polymers.

34. The coated coronary stent of claim 33, wherein said rapamycin-polymer coating has substantially uniform thickness and rapamycin in the coating is substantially uniformly dispersed within the rapamycin-polymer coating.

35. The coated coronary stent of claim 33 wherein the one or more resorbable polymers are selected from PLGA (poly(lactide-co-glycolide); DLPLA—poly(dl-lactide); LPLA—poly(1-lactide); PGA—polyglycolide; PDO—poly(dioxanone); PGA-TMC—poly(glycolide-co-trimethylene carbonate); PGA-LPLA—poly(1-lactide-co-glycolide); PGA-DLPLA—poly(dl-lactide-co-glycolide); LPLA-DLPLA—poly(1-lactide-co-dl-lactide); PDO-PGA-TMC—poly(glycolide-co-trimethylene carbonate-co-dioxanone) and combinations thereof.

36. The coronary stent of claim 33 wherein the polymer is 50/50 PLGA.

37. The coated coronary stent of claim 33, wherein said rapamycin is at least 50% crystalline.

38. The coated coronary stent of claim 1, wherein said polymer is a mixture of two or more polymers.

39. The coated coronary stent of claim 38, wherein said two or more polymers are intimately mixed,

40. The coated coronary stent of claim 33, wherein said stent provides an elution profile wherein about 10% to about 50% of rapamycin is eluted at week 1 after the composite is implanted in a subject under physiological conditions, about 25% to about 75% of rapamycin is eluted at week 2 and about 50% to about 100% of rapamycin is eluted at week 6.

41. The coated coronary stent of claim 33, wherein said stent provides an elution profile wherein about 10% to about 50% of rapamycin is eluted at week 1 after the composite is implanted in a subject under physiological conditions, about 20% to about 75% of rapamycin is eluted at week 2 and about 50% to about 100% of rapamycin is eluted at week 10.

42. The coated stent of claim 33, wherein the stent framework is a stainless steel framework.

43. A coated coronary stent, comprising:
a stent framework; and
a rapamycin-polymer coating wherein at least part of rapamycin is in crystalline form and wherein the polymer is bioabsorbable.

44. A coated coronary stent, comprising:
a stent framework; and
a macrolide immunosuppressive (limus) drug-polymer coating wherein at least part of the drug is in crystalline form and the polymer is bioabsorbable.

45. The coated, stent of claim 43, wherein the macrolide immunosuppressive drug comprises one or more of rapamycin, 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5″-Dihydroxypent-2′-en-1′-yl)-rapamycin 40-O-(2-Hydroxy)ethoxycar-bonylmethyl-rapamycin, 40-O-(3-Hydroxyl)propyl-rapamycin 40-O-(6-Hydroxyl)hexyl-rapamycin 40-O-[2-(2-Hydroxyl)ethoxy]ethyl-rapamycin 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin 40-O-(2-Nicotinoyloxy)ethyl-rapamycin, 40-O-[2-(N-Morpholino)acetoxy]ethyl-rapamycin 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxyl)ethyl-rapamycin, 28-O-Methyl-rapamycin, 40-O-(2-Aminoethyl)-rapamycin, 40-O-(2-Acetaminoethyl)-rapamycin 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′2′,3′-triazol-1′-yl)-ethyl]-rapamycin, 42-Epi-(tetrazolyl)rapamycin (tacrolimus), and 42-[3-hydroxy-2-(hydroxymethyl)-2-methylpropanoate]rapamycin (temsirolirnus).

46. The coated coronary stent of claim 43, wherein said macrolide immunosuppressive drug is at least 50% crystalline.

47. A method for preparing a coated coronary stent comprising the following steps:
providing a stainless or cobalt-chromium stent framework;
forming a macrolide immunosuppressive (limus) drug-polymer coating on the stent framework wherein at least part of the drug is in crystalline form and the polymer is bioabsorbable.

48. The method of claim 47 wherein the macrolide is deposited in dry powder form.

49. The method of claim 47 wherein the bioabsorbable polymer is deposited in dry powder form.

50. The method of claim 47 wherein the polymer is deposited by an e-SEDS process.

51. The method of claim 47 wherein the polymer is deposited by an e-RESS process.

52. The method of claim 47 further comprising sintering said coating under conditions that do not substantially modify the morphology of said macrolide.

53. The method of claim 47, wherein the macrolide immunosuppressive drug comprises one or more of rapamycin, 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl))benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin 40-O-(2-Hydroxy)ethoxycar-bonylmethyl-rapamycin, 40-O-(3-Hydroxyl)propyl-rapamycin 40-O-(6-Hydroxyl)hexyl-rapamycin 40-O-[2-(2-Hydroxyl)ethoxy]ethyl-rapamycin40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin 40-O-(2-Nicotinoyloxy)ethyl-rapamycin, 40-O-[2-(N-Morpholino)acetoxy]ethyl-rapamycin 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxyl)ethyl-rapamycin, 28-O-Methyl-rapamycin, 40-O-(2-Aminoethyl)-rapamycin, 40-O-(2-Acetaminoethyl)-rapamycin 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), and 42-[3-hydroxy-2-(hydroxymethyl)-2-methylpropanoate]rapamycin (temsirolimus).

54. The method of claim 47 wherein one or more resorbable polymers are selected from PLGA (poly(lactide-co-glycolide); DLPLA—poly(dl-lactide); LPLA—poly(1-lactide); PGA—polyglycolide; PDO—poly(dioxanone); PGA-TMC—poly(glycolide-co-trimethylene carbonate); PGA-LPLA—poly(1-lactide-co-glycolide); PGA-DLPLA—poly(dl-lactide-co-glycolide); LPLA-DLPLA—poly(1-lactide-co-dl-lactide); PDO-PGA-TMC—poly(glycolide-co-trimethylene carbonate-co-dioxanone).

55. A coated coronary stent, comprising:
a stent framework;
a first layer of bioabsorbable polymer; and
a rapamycin-polymer coating comprising rapamycin and a second bioabsorbable polymer wherein at least part of rapamycin is in crystalline form and wherein the first polymer is a slow absorbing polymer and the second polymer is a fast absorbing polymer.

56. The stent of claim 55 wherein the fast absorbing polymer is PLGA copolymer with a ratio of about 40:60 to about 60:40 and the slow absorbing polymer is a PLGA copolymer with a ration of about 70:30 to about 90:10.
